(19) United States
(12) Patent Application Publication
(10) Pub. No.: US 2008/0085326 A1
(43) Pub. Date: Apr. 10, 2008

(54) Antimicrobial material compositions enriched with different active oxygen species

(76) Inventor: Hai Xiong Ruan, Vancouver (CA)

Correspondence Address: Hai Xiong Ruan, #24 -1529 W 71st Avenue, Vancouver, BC, V6P 3B9, US

(21) Appl. No.: 11/538,647

(22) Filed: Oct. 4, 2006

(51) Int. Cl. A61K 33/38

(52) U.S. Cl. 424/618; 977/931; 977/906

(57) ABSTRACT

This invention discloses novel antimicrobial materials comprising of nanocrystalline metal, metal oxide, and active oxygen species in a permeable structure. It also discloses the compositions of the inventive material enriched with different active oxygen species. The inventive material showed a strong antimicrobial ability over a wide spectrum of microbies. The average log-reduction value was eight within five minutes. The inventive materials can remain sterile in air over more than two months. The methods used to prepare this inventive material are disclosed.

This application claims the benefit of U.S. Provisional Application No. 60/596271, filed on Oct. 4, 2005, the disclosure of which is incorporated herein by reference in its entirety.

### FIELD OF INVENTION

This invention is directed to antimicrobial materials comprising of porous biocompatible metal(s), metal oxide (same or different metals), and enriched different active oxygen species. Porous metal or metal oxide nanoparticles include the metal nanoparticles having or forming a porous structure or both. Active oxygen species sustaining in the porous structure include but not limited to O⁻, O₂²⁻, O_{b} and O₂⁻ species. Some of these species are enriched in one particular composition. Other species are enriched in a different composition of the said material.

A further aspect of the invention relates to a method for preparation of the said material containing different active oxygen species by modifying a composition of solid-state film of the metal complexes, which used to covert to the said material.

U.S. Patent Documents

| | | |
|---|---|---|
| 5935608 | Apr. 5, 1997 | Fujikawa, et al |
| 20040229034 | Nov. 18, 2004 | Djokic, Stojan |
| 5180585 | Jan. 19, 1993 | Jacobson, et al |
| 5753251 | May 19, 1998 | Burrel, et al |
| 5958440 | Sep. 28, 1999 | Burrell, et al. |
| 20040191423 | Sep. 30, 2004 | Ruan, Hai Xiong; et al. |
| 5534312 | Jul. 9, 1996 | Hill et al. |

### Cited References

1. Burrell, R. E., et al., Compositions and methods of metal-containing materials, in U.S. Pat. Appl. Pub/2004, (Can.). Us. p. 42 pp., Cont.-in-part of U.S. Ser. No. 277,673.
2. Pratt, A. S. and P. R. Smith, Antimicrobial compositions, in Eur. Pat. Appl. 1986, (Johnson Matthey PLC, UK). Ep. p. 21 pp.
3. Clement, J. L. and P. S. Jarret, Antibacterial silver. Metal-Based Drugs, 1994. 1(5-6): p. 467-82.
4. Imai, O., et al., Antibacterial silver film, its coated material, and coating process, in Jpn. Kokai Tokkyo Koho. 1997, (Nissin Electric Co., Ltd., Japan). Jp. p. 7 pp.
5. Shigeru, K., et al., Antibacterial, antifungal, and algicidal products and their manufacture, in Jpn. Kokai Tokkyo Koho. 1998, (Sumitomo Cement Co., Ltd., Japan). Jp. p. 7 pp.
6. Onozawa, Y., S. Shoshi, and S. Watanabe, Hard coated sheets with good transparency, in Jpn. Kokai Tokkyo Koho. 1999, (Lintec Co., Ltd., Japan). jp. p. 7 pp.
7. Miyata, S., Antifungal, antibacterial agents containing silver-, zinc-, or copper-exchanged aluminosilicates and their production method, in Jpn. Kokai Tokkyo Koho. 2000, (Kaisui Kagaku Kenkyusho K. K., Japan). Jp. p. 4 pp.
8. Ono, K., Antibacterial materials using titania and silver, in Jpn. Kokai Tokkyo Koho. 2002, (Tetra Pack Japan K. K., Japan). Jp. p. 5 pp.
9. Sakuma, S., et al., Antibacterial composite particles and antibacterial resin composition, in PCT Int. Appl. 2003, (Kabushiki Kaisha Sangi, Japan; Fuji Xerox Co., Ltd.). Wo. p. 36 pp.
10. Yan, J. and J. Cheng, Colloidal nanosilver antimicrobial solution, in PCT Int. Appl. 2003, (CC Technology Investment Co., Ltd., Peop. Rep. China; Soh, Kar Liang). Wo. p. 31 pp.
11. Liu, Y., Q. Xie, and W. Zhang, Antibacterial film and its preparation method, in Faming Zhuanli Shenqing Gongkai Shuomingshu. 2004, (Guangzhou Zhuodejia Film Co. Ltd., Peop. Rep. China). Cn. p. No pp. given.
12. Saravanapavan, P., et al., Antimicrobial macroporous gel-glasses: dissolution and cytotoxicity. Key Engineering Materials, 2004. 254-256(Bioceramics): p. 1087-1090.
13. Joshi, A. V., Anti-microbial handle system including active oxygen, ozone, halide or silver ion-releasing material, in U.S. Pat. Appl. Publ. 2005, (USA). Us. p. 10 pp.
14. Taylor, P. L., et al., Impact of heat on nanocrystalline silver dressings. Biomaterials, 2005. 26(35): p. 7230-7240.
15. Karandikar, B. M., B. L. Gibbins, and K. A. Cornell, Antimicrobial devices and compositions comprising silver, copper or zinc compounds, in PCT/nt. Appl. 2006, (Acrymed, Inc., USA). Wo. p. 77 pp.
16. Parsons, A. R. and D. W. Atkinson, Controlled release of antimicrobial agents into fluids, in PCT Int. Appl. 2006, (Bacstop Corporation Pty. Ltd., Australia). Wo. p. 45 pp.

### BACKGROUND OF THE INVENTION

The heavy metallic ions are known to have antimicrobial effects. The metallic ions include Ag, Au, Pt, Pd, Ir, Cu, Sn, Sb, Bi, and Zn. Among them, silver ion is best known with its antimicrobial properties. Inorganic silver compounds and organic silver compounds are well developed and used to prevent and treat microbial infection in modern medical practice. Most of them are silver salts. The short duration of some compounds and the need of re-applying these compounds frequently pose difficulty in practice.

U.S. Pat. No. 5,180,585 disclosed the compositions and preparation methods of antimicrobial coatings and some articles. They taught that the antimicrobial material is comprised of silver metal, copper compounds, and zinc compounds. The core and protective coatings were both prepared in aqueous solutions.

U.S. Pat. No. 5,753,251 disclosed a method to prepare anti-microbial coatings for medical device. The film deposited by physical evaporation creates atomic disorder in the structure of the deposited film. The film releases metal ions, atoms, molecules, or clusters of the anti-microbial metal when the film is in contact with an alcohol or a water based electrolyte. The physical evaporation method used to prepare atomic disordered silver film limits the quantity of the production and the size of the substances. The investment of the equipment is expensive in order to have a large-scale production. Another U.S. Pat. No. 6,939,568 is granted on Sep. 6, 2005 by a common inventor and others. This new patent disclosed an application of atomic disordered silver coating for inflammatory skin conditions. No new antimicrobial composition or preparation method was disclosed. Although they claimed that the biocompatible metal is silver in a composite form with oxygen, but no information of the antimicrobial composition was disclosed on the active oxygen species. No claims are made on silver and other metal composite with oxygen species that shows excellent antimicrobial effect. The oxygen or other gases used in preparation process were to create atomic disorder structure of the film, not to create active oxygen species. One other significant difference between their composition and this inventive material, their coatings or materials showed no antimicrobial effect after heating above 150 Celsius degree but this said material shows strong antimicrobial effect after the coating was heated at 300 Celsius for one hour. In addition, the killing rate of their film reported in the related literature is much slow than that of my inventive material. The killing mechanism taught in their invention is the releasing of atoms, ions, clusters, or molecules containing silver to a sufficient concentration. Their reported log reduction value after 30 minutes was about 5. However, the log-reduction value of the inventive material after five minutes has an average value of 8. The enhancement of antimicrobial activity from the said material by comprising different component such as different active oxygen species is obvious. Furthermore, in one of Burrel's publication, they found the silver oxide in a form of Ag₂O is a must component in the film in order to show any antimicrobial effect of their coating or film. In the said material, the silver oxide is in a form of Ag₂O₂ that is insoluble in water in contrast to that Ag₂O is soluble in water.

U.S. patent application 20040229034 disclosed material and composite material and their preparation methods for antimicrobial applications. Djokic et al claimed that the active oxidized silver species are the major component that response for antimicrobial effect. Djokic found that the essential factor leading to an antimicrobial activity of metallic silver is a presence of Ag oxides, which showed an antimicrobial activity. There is no active oxygen species documented in their research instead of “active silver species”.

The antimicrobial ability of the silver-containing coatings was generally believed that silver ions reacted with proteins by combing the —SH groups of enzymes, which leads the inactivation of the proteins. Another mechanism of antimicrobial of silver ions is believed that the silver ions affect DNA molecules by losing their replication abilities. All these proposed mechanism are directly related to silver ions or active silver ions, silver clusters et al. However, active oxygen species, some of them are radicals, are a better biocidal due to their strong oxidation and reduction ability that can instantly destroy the microbes through cellar lysis. Due to the short living time of these radicals, no solid-state material sustaining these radicals in a stable condition has been disclosed. Furthermore, the enrichment of few particular active oxygen species over other active oxygen species has never been disclosed in any publications.

US patent application 20040191423, Ruan et al. disclosed a silver/silver oxide film prepared from silver-containing material by irradiation. No claims in this application were made on antimicrobial ability of this coating. The composition of the said coating comprises of silver and silver oxide and no active oxygen species has been disclosed. The strong antimicrobial property of the said coating in this application related to active oxygen species has not been disclosed. Although this method disclosed to prepare a silver/silver oxide coating, the ability of tuning the amount of undisclosed active oxygen species in the coating has not been disclosed.

Other researchers reported oxygen species (O⁻, O₂²⁻, O_{b} and O₂⁻) absorbed on or dissolved in the metal or metal oxide surfaces under high oxygen pressure or at high activation temperature. The large amount of stable active oxygen species sustaining in a material under ambient conditions has not been disclosed. The porous structure of the metal and/or metal oxide composition used to sustain active oxygen species is first time disclosed in this invention. Although the reported active oxygen species can have antimicrobial effect according to their nature, the amount of these species and the process of producing these species reported in literature as cited above are questionable for any practicable antimicrobial applications. This could be the reason why no antimicrobial application of these active oxygen species on metal or metal oxide surfaces has been reported.

The partial oxidation of alkane related to some active oxygen species was greatly investigated by many researchers. However, how to enrich certain active oxygen species has not been disclosed. Furthermore, the ability to sustain different oxygen species on a support under ambient condition or high temperature has not been disclosed.

Metal and oxygen containing coating on the substrates shows 100% antimicrobial effect within minutes over a wide spectrum of microorganisms has not been reported in literature or disclosed in any patent or patent application. An antimicrobial coating comprising of porous silver and oxygen species (include O⁻, O₂²⁻, O₂⁻) prepared on various substrates under ambient condition is first time disclosed. The antimicrobial compositions comprising of biocompatible metal(s) and oxygen species (such like O⁻, O₂²⁻, O₂⁻) and optional other metal(s) or metal oxide(s) is first time disclosed. This aspect of the invention enables many methods to be developed to create such composition(s) for potential wide medical or public healthy applications.

### BRIEF DESCRIPTION OF THE DRAWINGS

FIG. 1 is a process flow diagram according to an embodiment of the invention;

FIG. 2 is an X-Ray diffraction pattern of an inventive coating on a silicon substrate.

FIG. 3 is a spectrum of auger electron microscopy of an inventive coating on a silicon substrate.

FIG. 4 is a scanning electron microscopy image of an inventive coating on a silicon substrate.

FIG. 5 is an X-ray photoelectron spectroscopy (XPS) spectrum of an inventive coating comprising silver and titanium oxide on a silicon substrate.

FIG. 6 is an O(1 s) core level X-ray photoelectron spectroscopy (XPS) spectrum of an inventive coating comprising silver and titanium oxide on a silicon substrate.

FIG. 7 is an O(1 s) core level X-ray photoelectron spectroscopy (XPS) spectrum of an inventive coating comprising only silver metal on a silicon substrate (with three resolved peaks).

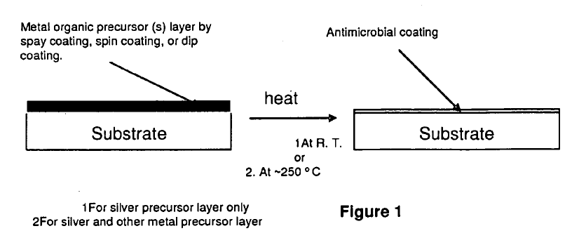

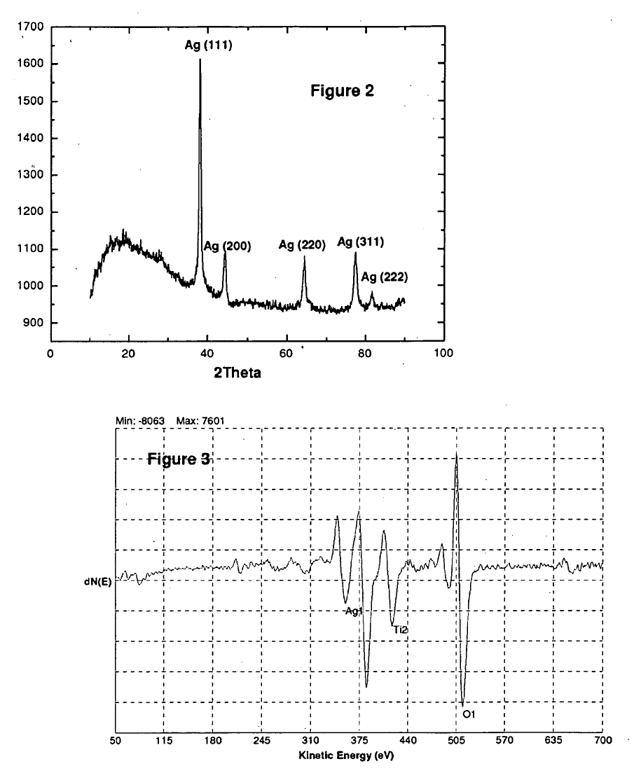

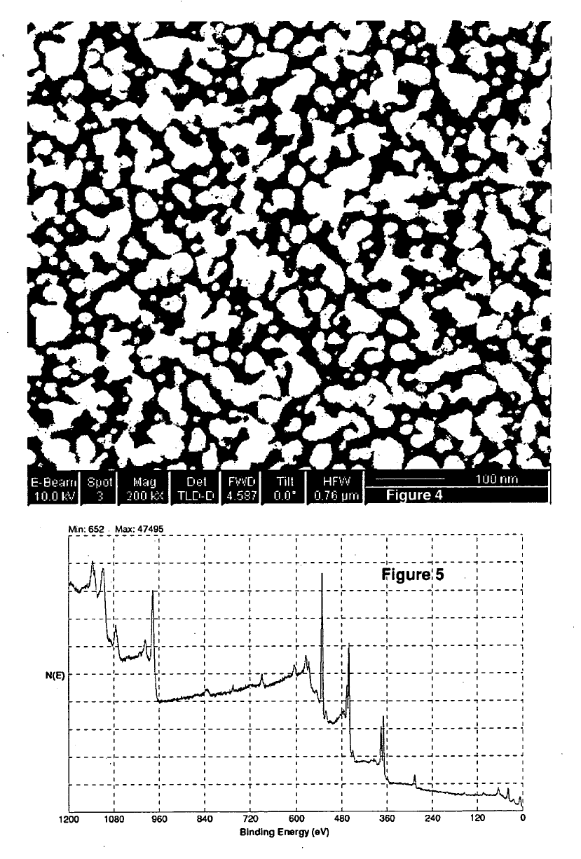

### SUMMARY OF THE INVENTION

The invention relates to antimicrobial material comprising of porous biocompatible metal(s), metal oxide(s), and various active oxygen species. The inventive coating provide strong microcidal ability within minutes on a wide spectrum of microorganisms, which include but not limited to Gram-negative and Gram-positive bacterial, Candida yeast, and Fungus. The biocompatible metal can choose from noble metal (Ag, Au, Pt, or others). The metal oxide can choose from oxides of transition metals, preferred to be Ti, Cu. The oxygen species includes but not limited to O⁻, O₂²⁻, O_{b} and O₂⁻. The coating comprising of polycrystalline silver nanoparticles and active oxygen species is porous. The porous structure of the metal coating is one of the keys to sustain different active oxygen species in the inventive coating.

Partial coverage of the substrate by the said material showed poor or no antimicrobial effect. It is believed that active oxygen species in the material lost or insufficient due to the limit of the porosity of the material is breached. It is hard to set a porosity limit for all the compositions since the content of the composition components and the compositions of the inventive material can be very different.

As stated in prior arts, a dense silver coating, or bulk material, such as silver sheet, silver rod, show no antimicrobial ability. A porous structure of metal and/or metal oxide is important to sustain active oxygen species in the inventive composition.

A great advantage of the inventive coating composition is self-sterilizing that the coating could kill the microorganisms instantly on the surface of the coating in a moisture environment or in their own survival fluid of the microorganisms when they contact the coating, or release the antimicrobial agents into the contact solution to kill the microorganisms. This property enables self-sterilization of the surface of the coated substances. A coating of the inventive composition could keep many medical devices, hospital rooms, public sanitation devices or areas, and others to be free of microorganisms' growth. For example, if the inventive coating is coated on the window screens or air filters, it can eliminate the microorganisms instantly to keep a healthy environment.

Another advantage of this invention is a method disclosed by Ruan, et al. to prepare the inventive antimicrobial material. An antimicrobial coating containing silver and active oxygen species can be prepared at room temperature from an ethanol solution of (1,1,1-trifluoroacetylacetono)silver(I) by spray coating, dip coating, or spin coating. This reaction of converting the solid-state film to the said coating can complete in minutes depending on the thickness of the precursor film. Such an economical and easy operational process can enable wide applications of this invention in many areas and fields.

Two types of antimicrobial coating compositions are disclosed in this invention. The first type comprises of only porous biocompatible metal (Ag, Au, and Pt, preferred to be Ag) and active oxygen species. The concentration of the metal precursor can vary from a concentration about 5% wt to a saturate concentration in one particular solvent (for example, about 20% wt in ethanol). The lower the precursor concentration is, the thinner the film thickness will be. Some partially coverage of the antimicrobial coating on the substrate could form when the precursor film is prepared from the precursor solution with a lower concentration. This should be avoided. For example, a suitable concentration of silver precursor in the solution to prepare a full coverage of the surface of the substrate is preferred to be greater than 5% wt.

The other type antimicrobial coating composition comprises of silver, other metal(s), preferred to be Ti, Cu, and active oxygen species. This type of coating has same strong antimicrobial effect as the first type coating over a wide spectrum of microorganisms. The second type coating composition can great reduce the amount of silver loading in the inventive coating. The silver precursor loading can be as low as 0.5% wt of titanium precursor and as high as 40% wt of titanium precursor. The different metal organic complexes that react with each other to form precipitates should be avoided.

When the other metal is titanium, this composition can increase the adhesion of the coating on the surface of substances. This is useful for the application requiring strong abrasion resistance and antimicrobial effect. When the ratio increases to high loading percentage (above ˜30%), silver nanoparticles formed on the top of the coating can be observed. One drawback of this is that the top silver layer has poor adhesion to the coating.

The transparency and color of the coating can be tuned by alternating the composition, or the ratio of the metal organic precursor(s), or the concentration of the metal organic precursor(s) solution. The 100% transparency of the antimicrobial coating can be achieved in visible light region easily with relative high titanium precursor loading. The color of the coating can be tuned from colorless to purple in multi-metal composite composition. For the coating comprising of silver and oxygen species, the appearance of the coating can vary from transparent to silvery reflective. Different substrates will also affect or alternate the color of the coating on the substrate. This could be due to the formation of the bonding between the deposited layer and the substrate at interface.

The disclosed fast preparation process for antimicrobial coatings enables many applications for medical device, medical treatment, and public health. The long lasting antimicrobial property extends the benefit to its application. The antimicrobial coating can be prepared on a bench-top or in a large-scale industrial manufacturing. This fast procedure also enables a way to recoat the surfaces of the substance as many times as needed. The long time effectiveness and easy maintenance of antimicrobial coating of the invention can be achieved as well.

Another advantage of the second type coating comprising silver, titanium, and active oxygen species is that the coating is acid resistive.

### DETAILED DESCRIPTION OF THE INVENTION

This invention relates to novel antimicrobial compositions comprising of at least one porous biocompatible metal, option of other metal(s), and active oxygen species (O⁻, O₂²⁻, O²⁻). The biocompatible metals can be noble metals, which are preferred to be Ag, Au, Pd, Pt, and Ir, or other metals preferred to be Cu, Sn, Sb, Bi, Zn, Ru, and Ni. The metal oxide can be noble metal oxides, or other metal oxides preferred to be TiO₂, CuO, Cu₂O, Ta₂O₅, SiO₂, NiO, Co₂O₃, and RuO, more preferred to be TiO₂, CuO, and Cu₂O. The metal oxide is normally used as a matrix to produce and sustain active oxygen species. One criteria of the metal oxide is that the metal oxide should be able to produce the active oxygen species and release these antimicrobial species when a desired porous structure with metal nanoparticles is formed.

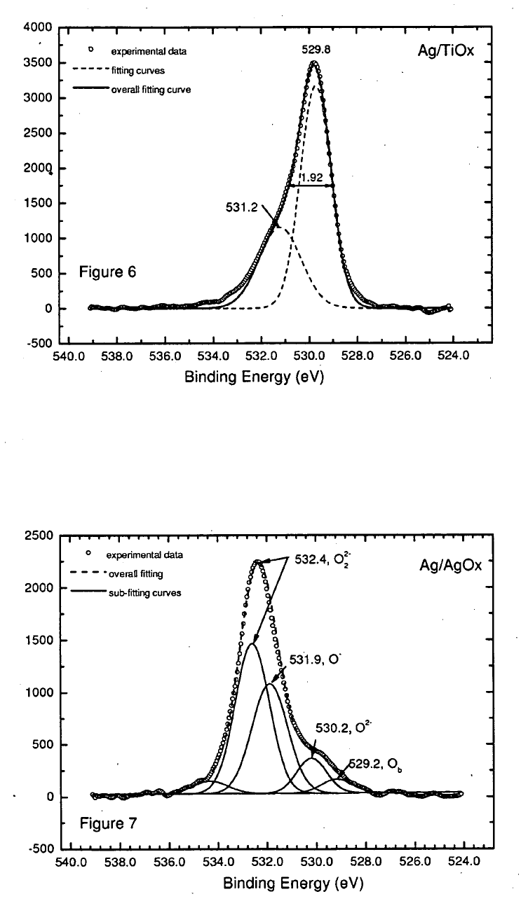

The active oxygen species were evident in oxygen 1 s core level X-ray photoelectron spectroscopy spectrum shown in FIGS. 6 and 7. By investigating the peak profile, different active oxygen species were identified. It is worth noting that the different active oxygen species enriched in different compositions of the inventive material. This also gives an opportunity to control the desired or preferred active oxygen species in the inventive compositions for potential catalytic reactions. This is first-time disclosure of preferred enrichment of some active oxygen species.

### Furthermore, a depth profile of a coating comprising of silver and oxygen by auger electron spectroscopy showed the consistent composition of the coating in depth.

In one embodiment of the inventive material, an antimicrobial coating comprising of nano-polycrystalline silver, silver oxide, and active oxygen species can be prepared by a single step on a glass substrate under ambient condition within minutes. One oxygen species having a binding energy 532.4 eV was enriched.

In another embodiment, an antimicrobial coating comprising of nano-polycrystalline silver, titania, and active oxygen species can be prepared by a single step on a glass substrate under 300 degree Celsius within minutes. One oxygen species having a binding energy 529.8 eV was enriched.

In one silver/silver oxide/active oxygen species embodiment, the inventive coating had a calculated porosity of 20% based on its Ellipsometry spectrum.

In one silver/silver oxide/active oxygen species embodiment, the inventive coating showed same strong antimicrobial effect after the film was heated at 300 Celsius degree for one hour.

Many methods could be used to prepare an antimicrobial coating having the said composition(s). For example, this antimicrobial composition could be prepared by exposing porous silver film in oxygen rich atmosphere, or produce porous silver film in oxygen rich atmosphere by other techniques, such as sol-gel methods, physical evaporation, or chemical vapor deposition method etc. In this invention, one preferred method is disclosed as described in the following.

The process for preparation the antimicrobial coating comprises of the following steps. The first step is to prepare an organic solution of metal organic precursor containing at lest one silver-containing precursor. The organic solvent can be a mixture of ethanol and 1-butanol. The metal organic precursor may be single chemical specie, or a mixture of different chemical compounds. If the different metal precursors are used to prepare an antimicrobial coating, the metal precursors should not react with each other to form precipitates. The solution of metal precursor(s) is used to prepare a coating on a substrate by spin coating, spray coating, or dip coating. The next step is to convert this coating under an oxygen-containing environment by irradiation, such as light, heat, or electron sources.

In an embodiment of the invention, if the initial precursor layer only contains (1,1,1-trifluoroacetylacetonato)silver(I), the layer can be converted to an antimicrobial coating under ambient conditions. The conversion rate of the precursor film can increase with increasing of the reaction temperature. The thickness of the deposited film can vary from several nanometers to hundreds of nanometers. The prepared antimicrobial film has a good adhesion to the substrate.

One advantage of this process is that the antimicrobial coating not only can be produced on the outer surface of one device, but also most inner surfaces of the substance can be coated homogeneously when the dip coating or spray coating technique is employed. The coating is also suitable to be prepared on the surface of cotton or synthetic fibers for potential antifungal or anti-odor application. Discoloring may appear on these surfaces due to the silver deposition. When the substrate is transparent, the coating can be tuned from transparent to a highly reflective surface by changing the concentration of the silver-containing material.

In the other embodiment of the invention, If the initial layer containing silver and titanium precursors, the coating can convert to the inventive material upon heating under 300 degree Celsius or by light irradiation under ambient conditions. The ratio between silver and titanium precursor can vary from 0.5% to 40% wt. The converted coatings with different silver contents showed same antimicrobial effects. However, different silver loading in the coating will change the coating appearance. This type of antimicrobial coating has several other benefits. The coating appears more strong abrasion resistance.

The metal and/or metal oxide is used as a matrix that can support silver and active oxygen species. If the metal oxide cannot maintain the oxygen species or react with active oxygen species, the coating could have very poor or no antimicrobial property. Slow annealing of a prepared inventive coating can lead to no antimicrobial at all by destroy the active oxygen species in the inventive material or changing the structure of the material. For example, after an inventive coating comprising of Ag/TiOx/active oxygen species was annealed at 35-Celsius degree for 1 hour with a heating rate of 5 degree Celsius per minute, the coating showed almost zero antimicrobial effect. This suggested that the annealing of an inventive coating could destroy the porous structure of the film or the active oxygen species or both. However, an inventive silver/silver oxide/active oxygen species coating maintains same strong antimicrobial effect after annealed under the same conditions.

In other embodiment of the invention, copper oxide is selected as a matrix. A copper organic precursor, Cu(II) trifluoroacetylacetonate, mixed with a silver precursor in an organic solvent. An inventive coating comprising of silver, copper, and active oxygen species produced from these metal-complexes shows excellent antimicrobial property as others.

Silver nanocrystals in antimicrobial coating has an average size ranging from few nanometers to several hundreds nanometers. The shapes of silver nanocrystals are non-spherical. The titanium (IV) oxide have a size ranging from few nanometers to tens nanometers. The least oxygen content in a silver-oxygen film was determined to be 7% by auger electron spectroscopy. It was found that the coating without silver loading, such as a TiO₂ coating, showed almost no antimicrobial effect.

Antimicrobial coating of the invention is ready to use for antimicrobial application. No further treatment, activation, or modification is needed. The inventive antimicrobial coating works in sterile water, physiological saline, and phosphate buffer solution.

The process for controlling microorganisms using antimicrobial coatings of the invention is provided in this invention. Microorganisms can be inhibited or killed in different media by contacting microorganisms with the antimicrobial coating in the media. Most media are aqueous media.

The two types of composition of the inventive coating were chosen as the following:
(1) The coating comprising of silver, silver oxide, and active oxygen species;
(2) The coating comprising of silver, copper or titanium oxide, and active oxygen species;

Glass slides and silicon chips with a size of 1″ by 1″ were used as the coating substrates. The metal precursors were selected from the followings: (1,1,1-trifluoroacetylacetono)silver(I), (1,1,1-trifluoroacetylacetono)copper(II), (mono-trifluoroacetylacetono)tris(iso-propoxo)Titanium(IV). The solvent is absolute alcohol or/and Methyl Isobutyl Ketone (MIBK).

The coating with composition 1 was prepared by following steps. A solution was prepared by dissolving (1,1,1-trifluoroacetylacetonato)silver(I) in an ethanol and 1-butanol (4:1 ratio in volume) mixture solvent. The coating was prepared by spin coating a substrate with this solution at 3000 rpm. The amount used to saturate the surface area of the substrate was used to prepare all the sample coatings. According to this, the coatings with identical thickness can be prepared from the same stock solution under the same spin coating condition. The layer of silver precursor on the substrate was then heated at 50 degree Celsius for 2 minutes on a hot plate. The prepared coating was stored in a sterile Petri dish before use.

The coating with composition 2 was prepared by following steps. A solution was prepared by dissolving (1,1,1-trifluoroacetylacetonato)silver(I) and Bis(1,1,1-trifluoroacetylacetonato)di(iso-propoxo)Titanium(IV) in ethanol and 1-butanol (4:1 ratio in volume) mixture solvent. Ethanol was anhydrous ethanol. A metal organic layer was prepared by spin coating a silicon substrate with the solution at 3000 rpm. This sample was heated at 250 degree Celsius for about 5 minutes on a hot plate. A coating comprising silver, titanium, and active oxygen species was prepared. The other solution was prepared by dissolving (1,1,1-trifluoroacetylacetonato)silver(I) and Bis(1,1,1-trifluoroacetylacetonato)copper(II) in MIBK. A metal organic layer was prepared by spin coating a silicon substrate with the solution at 3000 rpm. This sample was heated at 250 degree Celsius for about 5 minutes on a hot plate. A coating comprising silver, copper, and active oxygen species was prepared.

The coating of the invention shows strong antimicrobial effect over a wide spectrum of microorganisms. The coating has been tested with gram-positive and gram-negative bacterial, Fungus, and Candida Yeast. The gram-negative bacteria used in test were Escherichia Coli. The Gram-positive bacteria used In test were Staphylococcus Epidermidis. The fungal used in test was Aspergillus Fumigatus. The Candida Yeast used in test was Candida Tropicalis. Bacillus spores were tested with the inventive coatings as well. No viruses have been tested with the inventive coating.

The culture inoculums were prepared by following steps. The testing culture spread on a fresh medium plate was incubated at 37 degree Celsius overnight. One colony was transferred from the plate to 2.0 ml media solution (such as nutrient broth or Tryptic Soy broth depend on which culture is incubating) in a sterile test tube. This solution was incubated at 37 degree Celsius over night before use.

A general procedure employed to test the antimicrobial coating against microorganisms listed in Para 39 except fungal is described as following. First, the incubated culture was separated from the media solution by centrifuging at 3500 rpm for five minutes. The supernatant was decanted and a solid pellet of the culture was formed at the bottom of the test tube. This solid pellet was washed by sterile distilled water, physiological saline, or physiological phosphate buffer solution. The solution was vibrated at high speed for 30 seconds on a vortex device. Then the culture specie was separated from the solution by centrifuging. The supernatant was decanted. The culture pellet was washed twice before test. Appropriate dilutions of the culture were made with desired buffer solution (sterile water, physiological saline, or physiological phosphate buffer solution) before use. The testing slides including the slides with inventive antimicrobial coating (the coated slides) and the control slides with no antimicrobial coating were sterilized with 95% ethanol by flaming and cooled down to room temperature before use. 20 μl of the prepared culture solution was placed on each coated slide and two control slides. The solution was spread on the surface of the slides by an edge of a sterile cover glass. These slides with culture species were placed in a box, where some paper towels soaked with distilled water were placed at the bottom of the box to provide moisture environment for microorganism growth. The coated slides and control slides in the box were incubated at 37 degree Celsius over night. Each slide was then transferred into a 50.0 ml sterile tube filled with 10.0 ml sterile distilled water. The tube was vibrated at high speed for 20 seconds on a vortex device in order to wash the culture to the solution. The concentration of microorganism was determined by serial dilution of the solution and plating 100.0 μl of each solution on appropriate medium plate. Triplicate Petri plates (D100×15 mm) for each diluted solution were incubated at 37 degree Celsius room over night. Plates having colonies between 30 and 300 were counted. The concentration of the microorganism was determined from the mean of the plate counts. If the number of the colonies was below 30, all colonies were counted and the concentration of the tested microorganism was determined from the mean of the plate counts. If there is no colonies were observed, the colony count was said to be zero.

Antimicrobial activity was determined by log deduction, which has a formula:

D=log10(C₀)−log10(Cₜ)

Where CFU is the colony forming units; C₀ is the initial concentration of culture (CFU/ml); Cₜ is the concentration of culture at incubated time t; D is the log reduction. The concentration of the culture on the control slide after incubation was used as the initial concentration of the culture. For convenience, the value of D round to integer by rounding rules. If the digit at the first decimal place is less than five, the number rounds off. If the digit at first decimal place is equal or greater than 5, round to 1 to the left.

In case of the test with fungus, the culture was first collected from the growth plate by adding 10 ml surfactant solution (phosphate buffer solution-twin) into the plate due to the hydrophobic nature of the fungus. The culture solution was transferred in to a sterile tub. The cultures were separated from the solution by centrifuging. The fungus culture was washed with 5.0 ml sterile distilled water. Then the fungus was separated from the solution again. The black pellet was washed with sterile water twice more in order to remove possible surfactant residue. The pellet was dissolved in 2.0 ml sterile water and used to prepare different dilutions.

### EXAMPLE 1

Antimicrobial coatings comprising of silver and active oxygen species were prepared on different substrates according the procedure described in Para 44. A solution containing 10% wt (1,1,1-trifluoroacetylacetonato)silver(I) in ethanol and 1-butanol (4:1 ratio in volume) was prepared. The precursor layer was coated on two glass substrates (A1, A2) and one silicon (100) substrate-(S1). The samples, A1 and S1, were heated at 50 degree Celsius for 2 minutes. The other sample (A2) was heated at 250 degree Celsius for 1 minutes. Two blank glass slides were used as control for antimicrobial test. The culture used in test was Escherichia coli. The antimicrobial test was conducted according to the procedure described in Para 48. The result is listed in the following table.

| | Cₜ (CFU/ml) | Log reduction D |
|---|---|---|
| Ag/O coating, A1 | 0 | 9 |
| Ag/O coating, A2 | 0 | 9 |
| Ag/O coating, S1 | 0 | 9 |
| Control 1 | (1.1 ± 0.4)E9 | — |
| Control 2 | (1.3 ± 0.3)E9 | — |

From the test result, the samples with the inventive coating showed strong bactericidal effect. The coatings prepared at different temperature showed same strong bactericidal effect. So did the coatings prepared on different substrates. This provides great flexibility on process conditions and variety substrates for potential applications. The coating prepared at high temperature showed better adhesion than the one prepared at low temperature. The enhanced adhesion of the coating could be due to the bonding formation between the coating and the substrate.

### EXAMPLE 2

The antimicrobial coatings comprising of silver, titanium, and oxygen species were prepared according the procedure described in Para 45. A solution was prepared by dissolving (1,1,1-trifluoroacetylacetonato)silver(I) 0.0289 g and Bis(1,1,1-trifluoroacetylacetonato) di(iso-propoxo)titanium(IV) 0.1450 g in an ethanol and 1-butanol mixture solvent 1.5425 g. This solution was used as a stock solution. Metal precursors layer were prepared on two glass slides (labeled as F1 and F2) and two silicon (100) chips (labeled as S0 and S1). The coated samples were heated at 250 degree Celsius for 3 minutes on a hot plate. All the converted samples cooled to room temperature and kept in Petri dishes before test.

The antimicrobial test of the coating samples and controls (two blank cover slides, labeled as C1 and C2) was conducted according to the procedure described above in Para 48 with Escherichia Coli culture. The test result is listed in the following table. (where x is great than 2)

| | Cₜ (CFU/ml) | D |
|---|---|---|
| Ag/TiOx coating on | 0 | 9 |
| glass slide, F1 | | |
| Ag/TiOx coating on | 0 | 9 |
| glass slide, F2 | | |
| Ag/TiOx coating on a | 0 | 9 |
| silicon chip, S0 | | |
| Ag/TiOx coating on a | 0 | 9 |
| silicon chip, S1 | | |
| Control glass slide, C1 | (1.1 ± 0.4)E9 | — |
| Control glass slide, C2 | (1.3 ± 0.3)E9 | — |

The data indicated that the coated slides showed excellent bactericidal effect against Escherichia coli. Antimicrobial coating prepared on different substrates showed same antimicrobial ability.

### EXAMPLE 3

The antimicrobial coatings on glass substrate were prepared from the solutions containing silver and titanium precursor, in which the ratio between silver precursor and titanium precursor were 0.5%, 1%, 5%, 10%, 20%, and 30% in weight. Six different coatings were prepared on six glass slides according to the described procedure in Para 45. One blank glass slide was used as the control. Antibacterial test on these slides with Escherichia coli were conducted according the same procedure as described in Para 41. The test result is listed in the following table.

| | Cₜ (CFU/ml) | D |
|---|---|---|
| Ag/TiO2 Coating (0.5% wt) | 0 | 9 |
| Ag/TiO2 Coating (1.0% wt) | 0 | 9 |
| Ag/TiO2 Coating (5.0% wt) | 0 | 9 |
| Ag/TiO2 Coating (10.0% wt) | 0 | 9 |
| Ag/TiO2 Coating (20.0% wt) | 0 | 9 |
| Ag/TiO2 Coating (30.0% wt) | 0 | 9 |
| Control glass slide, C1 | (2.7 ± 0.5)E9 | — |

### The test results indicated that the coating with different silver loading showed same strong antibacterial effect against Escherichia coli. The least loading of silver in the coating of the invention has not been discovered.

### EXAMPLE 4

The slides used in Para 52 were sterilized with ethanol by flame and used to test Staphylococcus Epidermidis. Same test procedure described in [Para 50]was conducted except the medium plates used were Tryptic Soy agar plates. The test result was listed in the following table.

| | Cₜ (CFU/ml) | D |
|---|---|---|
| Ag/TiOx coating on glass | 0 | 8 |
| slide, F1 | | |
| Ag/TiOx coating on glass | 0 | 8 |
| slide, F2 | | |
| Ag/TiOx coating on a silicon | 0 | 8 |
| chip, S0 | | |
| Ag/TiOx coating on a silicon | 0 | 8 |
| chip, S1 | | |
| Control glass slide, C1 | (2.9 ± 0.3)E8 | — |
| Control glass slide, C2 | (1.4 ± 0.2)E9 | — |

### The data demonstrate strong efficiency of this invention coating against Staphylococcus Epidermidis.

### EXAMPLE 5

Antimicrobial coatings were tested with Fungus as well. The tested culture was Aspergillus Fumigatus. Two coatings (labeled as A3 and A4) comprising of silver, titanium and oxygen were prepared according the described process in Para 45. Two blank glass slides were used as the controls. The medium plates used in this test were MYPD agar plates. The test with fungal was followed the procedure described in Para 49. The test result is listed in the following table.

| | Cₜ (CFU/ml) | D |
|---|---|---|
| Ag/TiOₓ coating, A3 | 0 | 9 |
| Ag/TiOₓ coating, A4 | 0 | 9 |
| C1 | (3.0 ± 0.7)E8 | — |
| C2 | (3.6 ± 0.3)E9 | — |

### The data demonstrate strong efficiency of this invention coating against Aspergillus Fumigatus.

### EXAMPLE 6

The antimicrobial coatings were tested with Candida Yeast. The tested culture was Candida Tropicalis. The following coatings were prepared on glass slides according to the described procedure in Para 44 and 45. One coating (Ag1) comprised of silver and active oxygen species. One coating (ATO1) comprised of silver, titanium, and active oxygen species. Two coatings (T1, T2) comprised of only titanium and oxygen. The coating T2 was annealed at 350 degree Celsius for two hours at a heating rate of 10 degree Celsius. Two blank glass slides were used as the controls. The medium plates used in this test were the MYPD agar plates. The antimicrobial test was followed the procedure described in Para 48. The test result is listed In the following table.

| | Cₜ (CFU/ml) | D |
|---|---|---|
| Coating, Ag1 | 0 | 8 |
| Coating, ATO1 | 0 | 8 |
| Coating, T1 | (6.3 ± 0.2)E8 | 0 |
| Coating, T2 | (2.7 ± 0.8)E8 | 0 |
| Control 1 | (5.5 ± 1.5)E8 | — |

The data obtained from the coatings containing silver demonstrate strong antimicrobial effect against Candida Tropcalis. The coatings comprising of only titanium and oxygen showed no inhibition on the test culture. The annealed titanium oxide film showed no inhibition.

### EXAMPLE 7

The strength of antimicrobial activity of the inventive coatings was tested with Escherichia coli culture. The antimicrobial coatings were prepared separately according the procedures described in Para 44 and 45. A coating Ag1 was prepared from a solution containing 10% wt (1,1,1-trifluoroacetylacetonato)silver(I) in an alcohol solvent. The other coating Ag2 was prepared from a solution containing about 1% wt (1,1,1-trifluoroacetylacetonato)silver(I) in an alcohol solvent. Another coating ATO1 was prepared from a solution containing 5% mono-(trifluoroacetylacetonato) tri(iso-propoxo) titanium(IV) and comprised of 0.8% (1,1,1-trifluoroacetylacetonato)silver(I) in MIBK. A blank glass slide was used as a control, labeled as Control 1. The tested culture is Escherichia coli. The testing procedure was similar to the procedure described in Para 48 except the incubation time of the slides loaded with culture solution was five minutes. The solution used for dilution was physiological saline (0.9% wt NaCl sterile solution). The concentration of the coated slides and control slides were determined by the same procedure described in Para 48. The colonies in each plate were counted. The results showed in the table in following paragraph.

All slides except Ag2 were used for the test for different dilution solution. These slides were sterilized with 95% ethanol by flame. The antibacterial test of these slides were conducted according to the procedure described in Para 48 except the solvent used for dilution was physiological phosphate buffer solution (PBS). The concentration of the coated slides and the control slide were determined by the same procedure. The colonies in each plate were counted. The overall results were summarized in the following Table.

| | | 5 minutes | | 5 minutes |
|---|---|---|---|---|
| | | at 37° C. | | at 37° C. |
| | In NaCl(0.9%) | Log | In PBS | Log re- |
| | CFU | reduction D | CFU | duction D |
| Coating, | 0 | 7 | 0 | 9 |
| Ag1 | | | | |
| Coating, | 0 | 7 | 0 | 9 |
| ATO1 | | | | |
| Coating | (5.3 ± 1.0)E7 | 0 | — | — |
| Ag2 | | | | |
| Control 1 | (1.26 ± 0.14)E7 | 0 | (5.5 ± 1.0)E8 | 0 |

The above results indicated that antimicrobial coating had very strong microcidal ability against Escherichia coli within 5 minutes. Such an antimicrobial ability of the inventive solid coating is outstanding of other antimicrobial coatings disclosed in prior arts. This also suggested that antimicrobial coatings of the invention against other culture species could have same strength.
Different environment or solution did not decrease the antimicrobial ability of the coating. However, the discoloring of the coating with salt solutions was observed.
The coating labeled as Ag2 showed almost zero inhibition. This coating was prepared from a solution containing 1% of (1,1,1-trifluoroacetylacetonato)silver(I) in ethanol. A partial coverage of the silver film on the substrate was observed by Auger Electron Spectroscopy analysis. This partial coating of surface of the substrate could be the reason of this. This indicated that a minimum thickness and full coverage is needed in order to show strong antimicrobial effect of the inventive coating. A 5% of the silver precursor solution is preferred for preparation a coating comprising of silver and active oxygen species.

1. Antimicrobial materials comprise of metal(s) nanoparticles, and/or same or different metal oxide(s) nanoparticles, and active oxygen species in a permeable coating. The preferred metals are selected from Ag, Au, Pt, Pd, and Cu, more preferred is silver; the preferred metal oxides are selected from the oxides of Ag, Au, Pt, Pd, Cu, Ir, Sn, Sb, Bi, and Ti, the more preferred metal oxides are silver and titanium oxide. The active oxygen species includes but not limit to O⁻, O₂⁻, O₂²⁻, and O_{b}. The said materials show strong antimicrobial effect within minutes when they contact the microbies in solution. The antimicrobial effect shows an average value of log reduction number 8 over a wide spectrum of microbes. The said materials show self-sterilization ability for months in air.

2. The said material in [claim 1] wherein the permeable coating of metal or/and metal oxide nanoparticles could be in a porous or holey form.

3. The said material in [claim 1] has a porosity that is no less or greater than the limits of porosity of the said material that cannot sustain active oxygen species in the material.

4. The said material in [claim 1], in one particular composition comprising of silver (89% mol), and oxygen (11 mol %), has a porosity of about 20%.

5. The said material in [claim 1] comprises of at least one of active oxygen species.

6. The said material in [claim 1] wherein the most preferred metal is silver.

7. The said material in [claim 1] wherein the most preferred same metal oxide is silver oxide.

8. The said material in [claim 1] comprises of silver oxide in [claim 1], wherein the silver oxide includes polycrystalline silver (I,III) oxide] and/or silver (II, III) oxide.

9. The said material in [claim 1] wherein the most preferred different metal is copper when the other metal is silver.

10. The said material in [claim 1] wherein the most preferred different metal oxide is titanium oxide when the other metal is silver.

11. The said material comprises of titanium oxide in [claim 1], wherein the titanium oxide is in a form of Titanium (IV) dihydroxy oxide.

12. The said material comprises of titanium oxide in [claim 1], wherein the titanium oxide is in a form of Titanium (IV) dioxide.

13. The said material in [claim 1], wherein the active oxygen species are potential catalysts for partial oxidation reaction of alkane.

14. The metal in the said material in [claim 1], in a form of metallic silver has a grain size less than 200 nm.

15. The said material in [claim 1] comprising of only silver metal can have silver content 94 mol % in a composite with oxygen.

16. The said material in [claim 1] can have oxygen content 6 mol % in a composite with silver.

17. The said material in [claim 1], wherein active oxygen species can have different enriched oxygen species when the compositions of the said material are different.

18. The said material comprises of different-enriched active oxygen species in [claim 17], wherein one active oxygen species has a kinetic energy range between 530.0-532.5 eV when the said material in [claim 1] comprises of only one metal—silver.

19. The said material comprises different enriched active oxygen species in [claim 17], wherein one active oxygen species has a kinetic energy range between 529.0-531.5 eV when the said material in [claim 1] comprises of silver and titanium oxides.

20. The said material in [claim 1] comprising of silver, silver oxide, and active oxygen species has same strong antimicrobial effect after the said material was annealed at a temperature above 300 Celsius degree.
